(19) United States
(12) Patent Application Publication
(10) Pub. No.: US 2024/0096297 A1
(43) Pub. Date: Mar. 21, 2024

(54) DISPLAY DEVICE CONTROL FOR COLOR VISION DEFICIENCY COMPENSATION

(71) Applicant: HEWLETT-PACKARD DEVELOPMENT COMPANY, L.P., Spring, TX (US)

(72) Inventors: Hsiang-Ta Ke, Taipei City (TW); Yu-Ren Chen, Taipei City (TW); Chun-Feng Li, Taipei City (TW)

(21) Appl. No.: 17/932,465

(22) Filed: Sep. 15, 2022

(51) Int. Cl. G09G 5/02; A61B 3/032

(52) U.S. Cl. CPC G09G 5/02; A61B 3/032; G09G 2320/0666

(57) ABSTRACT

In some examples, a controller of a wearable device causes display by the wearable device of a test image, and adjusts a color property of the displayed test image. In response to an input provided by a user responsive to the displayed test image as the color property is adjusted, the controller determines a distribution of color wavelengths for an eye of the user, and detects a color vision deficiency of the user based on the determined distribution of color wavelengths. The controller provides control information to control a display device of the wearable device to compensate for the color vision deficiency.

### BACKGROUND

Color vision deficiency (CVD) refers to an inability of a user to distinguish certain colors, or even to see some colors. A human eye includes photoreceptor cells (also referred to as cone cells) in the retina of the eye. The cone cells include different classes of cone cells that are sensitive to photons of a corresponding different wavelengths. The wavelengths include a long wavelength (L), a middle wavelength (M), and a short wavelength (S) in the visible spectrum. Generally, L cone cells can detect the red color, M cone cells can detect the green color, and S cone cells can detect the blue color.

### BRIEF DESCRIPTION OF THE DRAWINGS

Some implementations of the present disclosure are described with respect to the following figures.

FIG. 1 Is a block diagram of a wearable device that is able to compensate for a user's color vision deficiency (CVD) according to some examples.

FIG. 2 is a graph that illustrates spectral waveforms corresponding to different colors, and a confusion range of wavelengths to be filtered for a user with CVD, in accordance with some examples.

FIG. 3 is a flow diagram of a process according to some examples.

FIG. 4 is a block diagram of a storage medium storing machine-readable instructions according to some examples.

FIG. 5 is a block diagram of a wearable device according to some examples.

FIG. 6 is a flow diagram of a process according to some examples.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

In the present disclosure, use of the term “a,” “an,” or “the” is intended to include the plural forms as well, unless the context clearly indicates otherwise. Also, the term “includes,” “including,” “comprises,” “comprising,” “have,” or “having” when used in this disclosure specifies the presence of the stated elements, but do not preclude the presence or addition of other elements.

Users with color vision deficiency (CVD) are unable to properly perceive color images displayed by display devices in electronic devices or colors of real world objects. When light of a certain wavelength reaches a retina of a human eye, cone cells are activated to allow the user to perceive colors. The different classes of cone cells (L, M, and S cells) of the eyes of users with CVD are either under activated or over activated, which can make it difficult for users which CVD to detect the true intended colors in color images displayed by display devices or of real-world physical objects.

In some cases, physicians or other health professionals may have to perform manual diagnoses of users with CVD so that treatment options can be manually developed by the professionals for the users. Such manual techniques can be time-consuming and expensive, since they involve visits to health professionals as well as purchase of devices such as special eyeglasses or contact lenses to treat the CVD when possible.

In accordance with some implementations of the present disclosure, automated techniques or mechanisms are provided by a wearable device to compensate for CVD of a user that wears the wearable device. Examples of wearable devices can include any or some combination of the following: a head-mounted device, electronic eyeglasses, or any other type of electronic device that can be worn by a user and that can include a collection of display devices (a single display device or multiple display devices) that is viewable by the eye(s) of the user.

The wearable device can cause display of a test color pattern, adjust a color property (e.g., a hue) of the displayed test color pattern, and determine a distribution of color wavelengths (based on measurements by color sensors in the wearable device) for an eye of the user in response to an input provided by a user responsive to the displayed test color pattern as the color property is adjusted. The wearable device can detect a CVD of the user based on the determined distribution of color wavelengths, and can provide control information to control a display device of the wearable device to compensate for the CVD. As examples, the wearable device includes an adjustable color filter for each display device that can be adjusted to filter out (remove or attenuate) color wavelengths in a target wavelength range (referred to as the “confusion range” in the present discussion) to compensate for the CVD. As further examples, a controller in the wearable device can adjust a color property (e.g., a hue) of displayed images in the display device(s) to compensate for the CVD.

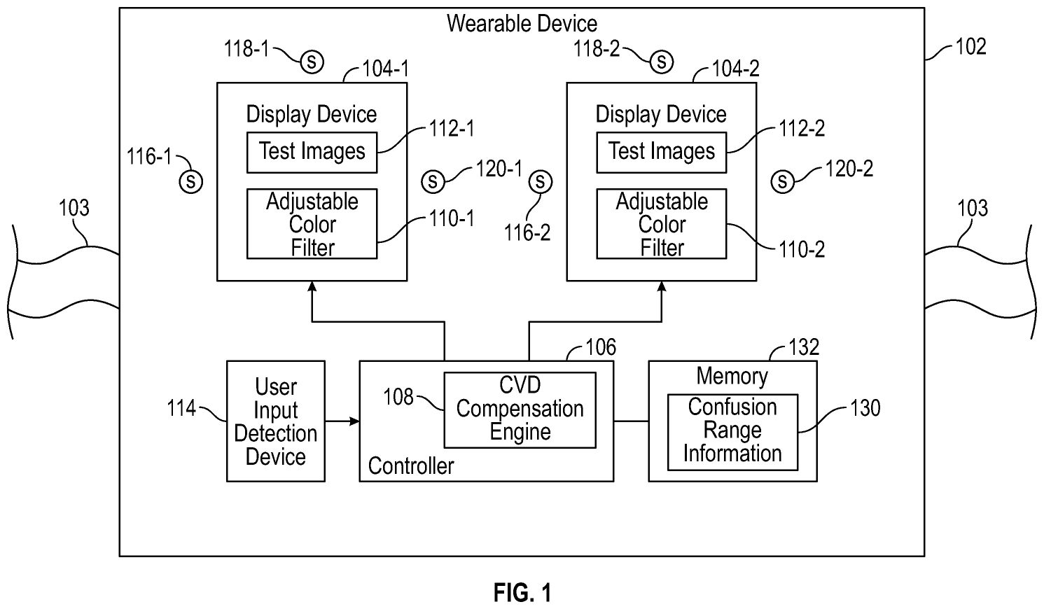

FIG. 1 is a block diagram of a wearable device 102 that can be worn by a user, such as the user's head. For example, the wearable device 102 can have attachment members 103 (e.g., straps, eyeglass temples, etc.).

The wearable device 102 includes display devices 104-1 and 104-2. The display device 104-1 is to display images for a left eye of the user when the wearable device 102 is worn by the user, and the display device 104-2 is to display images for a right eye of the user when the wearable device 102 is worn by the user. In other examples, instead of having two display devices, the wearable device 102 can include a single display device, which can be viewed by either or both of the eyes of the user.

The wearable device 102 includes a controller 106 that can be provided within a housing of the wearable device 102. As used here, a “controller” can refer to a hardware processing circuit, which can include any or some combination of a microprocessor, a core of a multi-core microprocessor, a microcontroller, a programmable integrated circuit, a programmable gate array, or another hardware processing circuit. Alternatively, a “controller” can refer to a combination of a hardware processing circuit and machine-readable instructions (software and/or firmware) executable on the hardware processing circuit.

The controller 106 includes a CVD compensation engine 108 according to some implementations of the present disclosure. The CVD compensation engine 108 can be implemented with a portion of the hardware processing circuit of the controller 106, or alternatively, can be implemented using machine-readable instructions executable by the controller 106.

In accordance with some implementations of the present disclosure, the display device 104-1 includes an adjustable color filter 110-1, and the display device 104-2 includes an adjustable color filter 110-2. The adjustable color filters 110-1 and 110-2 are controllable by the CVD compensation engine 108. The adjustable color filters 110-1 and 110-2 are controlled by the CVD compensation engine 108 to filter out (remove or attenuate) wavelengths in a confusion range of a color spectrum.

A “color spectrum” refers to the spectrum of wavelengths corresponding to visible light. The “confusion range” of the color spectrum refers to a range of wavelengths in the color spectrum that a user with CVD is unable to properly perceive. The confusion range of the color spectrum for a given user with CVD (who is wearing the wearable device 102) can be determined by the CVD compensation engine 108 based on test images 112-1 and 112-2 displayed by the display devices 104-1 and 104-2, respectively. The test images 112-1 and 112-2 are presented by the CVD compensation engine 108.

The test images 112-1 and 112-2 can each include a series of test images in which the hue is modified between each successive test image. Each of the series of tests images contains a target pattern (e.g., a specific numeral, a specific character, a specific image object, etc.) that would be visible to a person without CVD but that may be difficult for a user with CVD to see. The test images in the series of test images are identical to one another except that a color property (such as hue and possibly another color property) of the target pattern is varied among the test images. Each test image can include the target pattern and a background pattern around the target pattern. The hue of the target pattern in the test image may be varied across different test images, while the hue of the background pattern remains unchanged.

The term “hue” can refer to a basic tone of color or a root (or dominant) color. Thus, adjusting the hue refers to changing the root (dominant) color of the target pattern.

By adjusting the hue of the target pattern in the successive test images, the user with CVD may be able to perceive the target pattern in a subset of the test images. For example, assume that the series of test images (e.g., test images 112-1 or 112-2) includes test image 1, test image 2, . . . , test image N (N≥2) with target patterns of different hues. When test image 1 is presented to the user with CVD in the display device 104-1 or 104-2, the user may be unable to see the target pattern in the test image 1. This may be true in other test images of the series of test images. When test image j (j selected from 1 to N) is displayed with a respective hue, the user with CVD may see the test pattern, at which point the user can provide an input to indicate that the test pattern is visible to the user. In some examples, the hue in the series of test images can be varied from low to high for the green color and/or the red color.

In some examples, each eye of the user can be tested independently of one another. Thus, the series of tests images 112-1 can be presented in the display device 104-1, while the display device 104-2 remains off, to test the left eye of the user. On the other hand, to test the right eye of the user, the series of test images 112-2 can be presented in the display device 104-2, while the display device 104-1 remains off.

When the user sees the specified target in the series of test images 112-1 or 112-2, the user can provide a pattern detected input, which can be in the form of a gesture, an activation of a user input device, and so forth. The wearable device 102 includes a user input detection device 114 that is able to detect the user's pattern detected input. For example, the wearable device 102 can include a camera or another sensor to detect a user gesture. A specific gesture made by the user, such as by raising the user's hand or finger(s), waving of the user's hand, etc., can be detected by the user input detection device 114. The detected user input is provided to the controller 106, which can recognize the detected user input as being a positive indication of the user's perception of the specified target in a given test image of the series of test images. As another example, the user can provide the input using a user input device such as a keyboard, a mouse, a touchscreen, and so forth. The user input detection device 114 can be coupled to the user input device over a cable or wirelessly to receive signals from the user input device when the user input device is manipulated by the user to provide the user's pattern detected input.

In some examples, each of the display devices 104-1 and 104-2 is associated with a group of color sensors. The display device 104-1 is associated with a group of color sensors 116-1, 118-1, and 120-1. The color sensors 116-1, 118-1, and 120-1 can be red, green, and blue color sensors, respectively, to detect the red, green, and blue colors, respectively.

Similarly, the display device 104-2 is associated with a group of color sensors 116-2, 118-2, and 120-2, to detect the red, green, and blue colors, respectively. The group of color sensors 116-1, 118-1, and 120-1 is used to sense colors in a test image 112-1 displayed by the display device 104-1, and the group of color sensors 116-2, 118-2, and 120-2 is used to detect colors in a test image 112-2 displayed by the display device 104-2.

In other examples, one group of color sensors can be used to sense colors in test images in both the display devices 104-1 and 104-2.

The group of color sensors 116-1, 118-1, and 120-1 takes color measurements of a test image 112-1 (one of the series of test images 112-1) in the display device 104-1 in response the user indicating that the user is able to perceive the target pattern (based on the user's pattern detected input discussed above). Similarly, the group of color sensors 116-2, 118-2, and 120-2 takes color measurements of a test image 112-2 (one of the series of test images 112-2) in response the user indicating that the user is able to perceive the target pattern.

Based on the color measurements performed by the group of color sensors 116-1, 118-1, and 120-1, the CVD compensation engine 108 can produce curves representing color wavelengths perceivable by the left eye of the user. Similarly, based on the color measurements performed by the group of color sensors 116-2, 118-2, and 120-2, the CVD compensation engine 108 can produce curves representing color wavelengths perceivable by the right eye of the user.

The CVD compensation engine 108 is aware where the test pattern is located in each test image (e.g., the CVD compensation engine 108 may be configured with information of the specific arrangement of pixels of each test image that contains the test pattern). Based on the color measurements of the target pattern relative to the color measurements of the background pattern, the CVD compensation engine 108 is able to determine the color wavelengths detectable by the S, M, and L cone cells of the human eye. For example, empirical data may be collected that correlate specific color measurements to corresponding wavelength ranges of the S, M, and L cone cells. The empirical data can be stored in mapping information, for example, that can map color measurements (measurements of hues) from the group of color sensors (116-1, 118-1, and 120-1 or 116-2, 118-2, and 120-2) to respective color wavelength ranges of the S, M, and L cone cells.

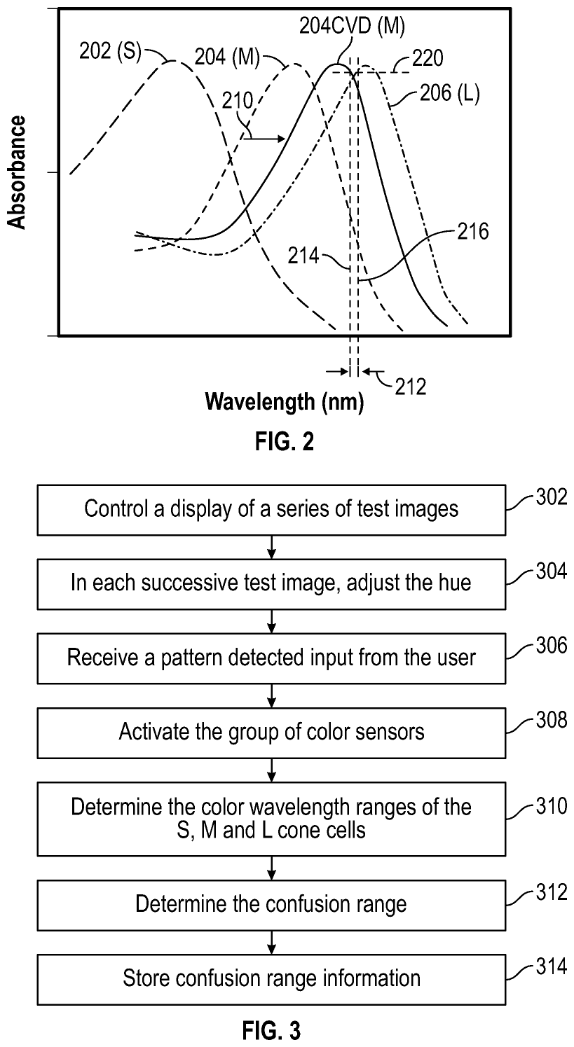

FIG. 2 is a graph depicting examples of color wavelengths that can be detected by S, M, and L cone cells of a human eye. The horizontal axis of the graph represents wavelength (in nanometers or nm, for example), and the vertical axis of the graph represents an absorbance of cone cells (corresponding to quantity of light detected by the cone cells).

The S cone cells are activated by color wavelengths (corresponding to the blue color) represented by a curve (or equivalently, a spectral waveform) 202, the M cone cells (of a human eye without CVD) are activated by color wavelengths (corresponding to the green color) represented by a curve 204, and the L cone cells are activated by color wavelengths (corresponding to the red color) represented by a curve 206.

However, for a user with CVD, the M cone cells may not detect the green color properly. For example, the M cone cells of a human eye with CVD are activated by color wavelengths represented by a curve 204CVD, which is shifted to the right (e.g., by about 30 nm or another amount) with respect to the curve 204, as indicated by an arrow 210. As a result, the M cone cells and the L cone cells of the human eye with CVD are activated by color wavelengths that are relatively close to one another (this results due to the M cone cells being under activated). The gap between the peak of the curve 204CVD and the curve 206 is less than the gap between the peak of the curve 204 and the curve 206. As a result, the human eye with CVD may perceive smaller differences between the red, orange, yellow, and green colors.

The curves 202, 204CVD, and 206 are derived by the CVD compensation engine 108 based on color measurements made by the group of color sensors (116-1, 118-1, and 120-1 or 116-2, 118-2, and 120-2).

In other examples, instead of or in addition to the curve 204 corresponding to the M cone cells shifting right, the curve 206 corresponding to the L cone cells may shift to the left (this results due to the L cone cells being over activated), which similarly results in the gap between the peak of the curve representing color wavelengths detectable by the M cone cells and the peak of the curve representing color wavelengths detectable by the L cone cells being reduced.

Due to the reduced gap between the curve 204CVD and the curve 206 representing respectively the color wavelengths detectable by the M and L cone cells of a human eye with CVD, a confusion range 212 results. The confusion range 212 of the color spectrum refers to a range of wavelengths in the color spectrum that the human eye with CVD is unable to properly detect. The confusion range 212 can be defined as a range that starts at a first wavelength (represented by dashed line 214) and ends at a second wavelength (represented by dashed line 216).

To identify the confusion range 212, the CVD compensation engine 108 can look at each 10-nm wavelength range across the color spectrum represented by the horizontal axis of the graph shown in FIG. 2. In each 10-nm wavelength range, the CVD compensation engine 108 can determine whether a distance between a peak portion of a first curve (any of 202, 204CVD, and 206) and a peak portion of a second curve (any other one of 202, 204CVD, and 206) is less than a specified distance threshold. If so, that indicates that the gap between the first curve and the second curve is too low, which indicates that CVD is present.

A “peak portion” of a curve is the portion where absorbances of the curve exceeds a specified absorbance threshold, as represented by a horizontal dashed line 220 in FIG. 2. An absorbance of the curve 204CVD or 206 above the specified absorbance threshold means an activation of the M cone cells or R cone cells, respectively, above an activation threshold. Thus, in the 10-nm wavelength range represented by 212, it is detected that the distance between the peak portion of the curve 204CVD and the peak portion of the curve 206 is less than the specified distance threshold. Activation of the M cone cells and R cone cells in response to colors represented by the wavelengths in the confusion range 212 will result in a user being unable to accurately perceive colors in a displayed image or colors of a real object.

In other examples, instead of testing using 10-nm wavelength ranges, wavelength ranges of different widths (greater than 10 nm or less than 10 nm) can be used.

The specified distance threshold can be based on a mean distance between peak portions of the different curves 202, 204CVD, and 206 (e.g., 30% or some other percentage of the mean distance). Thus, if in a current 10-nm wavelength range being considered, the distance between the peak portion of the first curve and the peak portion of the second curve is less than 30% of the mean distance, then the current 10-nm wavelength range can be identified as being part of the confusion range.

FIG. 3 is a flow diagram of a process of determining a confusion range for each eye of a user. The process can be performed by the CVD compensation engine 108, for example.

The CVD compensation engine 108 controls (at 302) a display of a series of test images on a respective display device (104-1 or 104-2). In each successive test image of the series of test images, the process 300 includes adjusting (at 304) the hue of the successive test image such that the hue of the successive test image is different from a prior displayed test image. The successive test images are displayed until the eye of the user currently being tested sees the target pattern in the test images.

The CVD compensation engine 108 receives (at 306) a pattern detected input from the user, which is provided by the user when the user sees the target pattern in the currently displayed test image.

The CVD compensation engine 108 activates (at 308) the group of color sensors (116-1, 118-1, 120-1 or 116-2, 118-2, 120-2) to take color measurements of the test image in which the user saw the target pattern.

The CVD compensation engine 108 determines (at 310) the color wavelength ranges of the S, M, and L cone cells based on the sensor measurements from the color sensors. Based on the color wavelength ranges of the S, M, and L cone cells (and especially the color wavelength ranges of the M and L cone cells), the CVD compensation engine 108 determines (at 312) the confusion range 212 for the current eye of the user. The CVD compensation engine 108 stores (at 314) confusion range information 130 representing the confusion range 212 in a memory 132. The confusion range information 130 can specify the first wavelength (214) and the second wavelength (216) that define the confusion range 212.

The memory 132 can be implemented with a collection of memory devices (a single memory device or multiple memory devices). A memory device can include any or some combination of a dynamic random access memory (DRAM) device, a static random access memory (SRAM) device, a flash memory device, and so forth.

The process of FIG. 3 can be reiterated for the other eye of the user to test if the other eye also experiences CVD, and if so, to store confusion range information for the other eye.

In some examples, the CVD compensation engine 108 can perform the following tasks to compensate for CVD of a human eye. For example, the CVD compensation engine 108 can adjust the adjustable color filter 110-1 or 110-2 to filter out (remove or attenuate) light with wavelengths within the confusion range 212. In some examples, each adjustable color filter 110-1 or 110-2 includes an electrochromic lens that can be controlled to filter out certain wavelengths. An electrochromic lens includes cholesterol liquid crystals that are optically polarizable. A long axis of the cholesterol liquid crystals can twist to different angles in response to an applied electric field. The cholesterol liquid crystals can pass light of specified wavelengths at different angles of the long axis of the cholesterol liquid crystals. Controlling the twisting of the cholesterol liquid crystals at a high frequency (higher than detectable by the human eye) can allow the CVD compensation engine 108 to control what wavelengths are passed through the electrochromic lens and what wavelengths are not (such as the wavelengths in the confusion range 212).

In other examples, other types of adjustable color filters can be used to filter out wavelengths in the confusion range 212, under control of the CVD compensation engine 108.

In further examples, in addition to or instead of controlling the adjustable color filters 110-1 and 110-2 to filter out wavelengths in the confusion range 212, the CVD compensation engine 108 can adjust the hue of images displayed by the display device 104-1 and/or 104-2. The adjustment of the hue of an image displayed by a display device can depend upon where the confusion range 212 occurs.

For example, if the confusion range 212 is greater than 650 nm but less than 700 nm, then the red hue is adjusted by the CVD compensation engine 108. For example, the CVD compensation engine 108 can increase the red hue by 30% in the displayed image.

As another example, if the confusion range 212 is greater than 450 nm but less than 650 nm, then the green hue is adjusted by the CVD compensation engine 108. For example, the CVD compensation engine 108 can increase the green hue by 30% in the displayed image, which can cause the green color to approach the yellow color, for example.

As a further example, if the confusion range 212 is greater than 400 nm but less than 450 nm, then the blue hue is adjusted by the CVD compensation engine 108. For example, the CVD compensation engine 108 can increase the blue hue by 30% in the displayed image, which can cause the blue color to approach the cyan color, for example.

In further examples, the CVD compensation engine 108 can adjust the lightness property of an image displayed in a display device to change the color contrast.

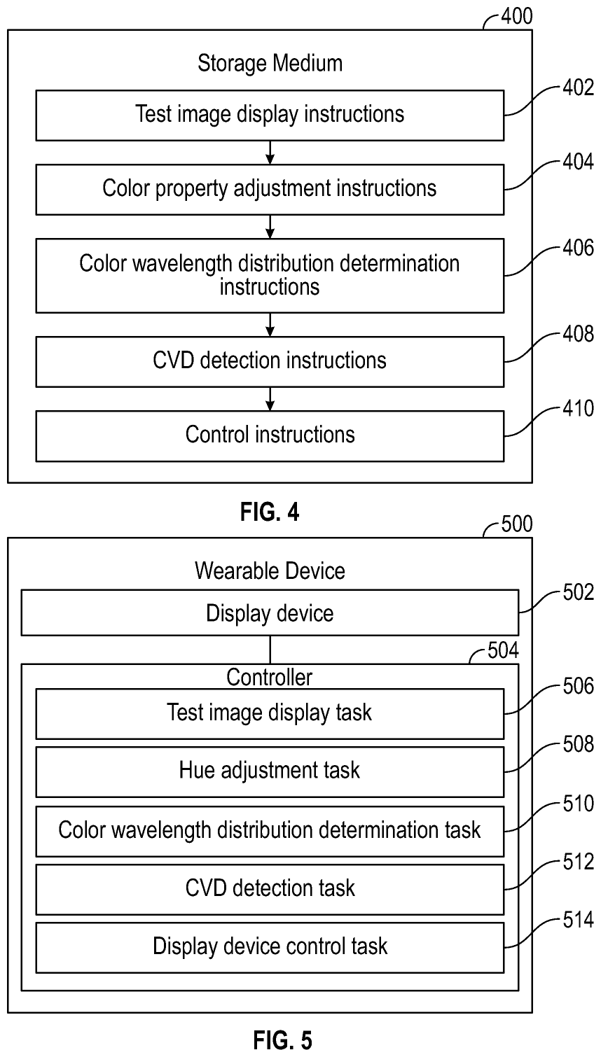

FIG. 4 is a block diagram of a non-transitory machine-readable or computer-readable storage medium 400 storing machine-readable instructions that upon execution cause a controller of a wearable device (e.g., 102 in FIG. 1) to perform various tasks.

The machine-readable instructions include test image display instructions 402 to cause display by the wearable device of a test image (e.g., the series of test images 112-1 or 112-2).

The machine-readable instructions include color property adjustment instructions 404 to adjust a color property of the displayed test image. For example, the adjusted color property can be the hue of the displayed test image, such as the hue corresponding to the green color and/or the red color.

The machine-readable instructions include color wavelength distribution determination instructions 406 to, in response to an input provided by a user responsive to the displayed test image as the color property is adjusted, determine a distribution of color wavelengths for an eye of the user. In some examples, the input provided by the user responsive to the displayed test image as the color property is adjusted includes an indication that a target pattern in the displayed test image is visible to the user.

In some examples, determining the distribution of color wavelengths for the eye of the user is based on detecting a given hue at which the target pattern in the displayed test image is indicated as visible to the user.

The machine-readable instructions include CVD detection instructions 408 to detect a CVD of the user based on the determined distribution of color wavelengths.

The machine-readable instructions include control instructions 410 to provide control information to control a display device of the wearable device to compensate for the CVD.

In some examples, the distribution of color wavelengths includes a first curve representing wavelengths detectable by S cone cells of the eye of the user, a second curve representing wavelengths detectable by M cone cells of the eye of the user, and a third curve representing wavelengths detectable by L cone cells of the eye of the user.

In some examples, detect the CVD of the user is based on identifying that a separation between peak portions of green and red spectral waveforms of the determined distribution of color wavelengths is less than a distance threshold.

FIG. 5 is a block diagram of a wearable device 500 including a display device 502 and a controller 504. The controller 504 is able to perform various tasks.

The tasks of the controller 504 include a test image display task 506 to cause display of a test image in the display device 502.

The tasks of the controller 504 include a hue adjustment task 508 to adjust a hue of the displayed test image.

The tasks of the controller 504 include a color wavelength distribution determination task 510 to, in response to an input provided by a user responsive to the displayed test image as the hue is adjusted, determine a distribution of color wavelengths for an eye of the user. The distribution of color wavelengths includes a first spectral waveform for a green color detectable by the eye of the user as indicated by the input, and a second spectral waveform for a red color detectable by the eye of the user as indicated by the input.

The tasks of the controller 504 include a CVD detection task 512 to detect a CVD of the user based on a distance between the first spectral waveform and the second spectral waveform.

The tasks of the controller 504 include a display device control task 514 to control the display device of the wearable device to compensate for the CVD.

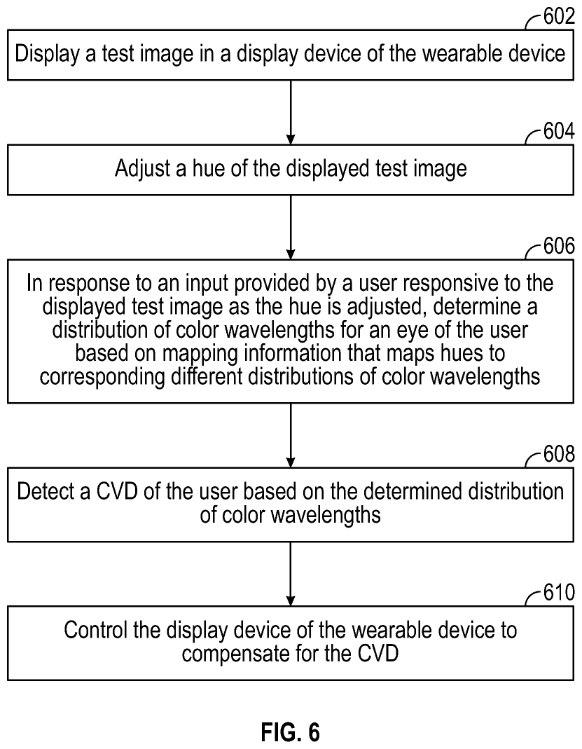

FIG. 6 is a flow diagram of a process according to some examples, which can be performed by a wearable device (e.g., 102 in FIG. 1).

The wearable device displays (at 602) a test image in a display device of the wearable device. The test image can be part of a series of test images.

The wearable device adjusts (at 604) a hue of the displayed test image. The hue of each successive test image of the series of test images can be different from a prior test image of the series of test images.

In response to an input provided by a user responsive to the displayed test image as the hue is adjusted, the wearable device determines (at 606) a distribution of color wavelengths for an eye of the user based on mapping information that maps hues to corresponding different distributions of color wavelengths.

The wearable device detects (at 608) a CVD of the user based on the determined distribution of color wavelengths.

The wearable device controls (at 610) the display device of the wearable device to compensate for the CVD.

A storage medium (e.g. 400 in FIG. 4) can include any or some combination of the following: a semiconductor memory device such as a DRAM or SRAM, an erasable and programmable read-only memory (EPROM), an electrically erasable and programmable read-only memory (EEPROM) and flash memory or other type of non-volatile memory device; a magnetic disk such as a fixed, floppy and removable disk; another magnetic medium including tape; an optical medium such as a compact disk (CD) or a digital video disk (DVD); or another type of storage device. Note that the instructions discussed above can be provided on one computer-readable or machine-readable storage medium, or alternatively, can be provided on multiple computer-readable or machine-readable storage media distributed in a large system having possibly plural nodes. Such computer-readable or machine-readable storage medium or media is (are) considered to be part of an article (or article of manufacture). An article or article of manufacture can refer to any manufactured single component or multiple components. The storage medium or media can be located either in the machine running the machine-readable instructions, or located at a remote site from which machine-readable instructions can be downloaded over a network for execution.

In the foregoing description, numerous details are set forth to provide an understanding of the subject disclosed herein. However, implementations may be practiced without some of these details. Other implementations may include modifications and variations from the details discussed above. It is intended that the appended claims cover such modifications and variations.

What is claimed is:

1. A non-transitory machine-readable storage medium comprising instructions that upon execution cause a controller of a wearable device to:
cause display by the wearable device of a test image;
adjust a color property of the displayed test image;
in response to an input provided by a user responsive to the displayed test image as the color property is adjusted, determine a distribution of color wavelengths for an eye of the user;
detect a color vision deficiency of the user based on the determined distribution of color wavelengths; and
provide control information to control a display device of the wearable device to compensate for the color vision deficiency.

2. The non-transitory machine-readable storage medium of claim 1, wherein the adjusting of the color property of the displayed test image comprises adjusting a hue in the displayed test image.

3. The non-transitory machine-readable storage medium of claim 2, wherein the adjusting of the hue of the color in the displayed test image comprises adjusting a hue corresponding to a green color or a red color in the displayed test image.

4. The non-transitory machine-readable storage medium of claim 1, wherein the input provided by the user responsive to the displayed test image as the color property is adjusted comprises an indication that a target pattern in the displayed test image is visible to the user.

5. The non-transitory machine-readable storage medium of claim 4, wherein the adjusting of the color property of the displayed test image comprises adjusting a hue in the displayed test image, and wherein the instructions upon execution cause the controller of the wearable device to:
determine the distribution of color wavelengths for the eye of the user based on detecting a given hue at which the target pattern in the displayed test image is indicated as visible to the user.

6. The non-transitory machine-readable storage medium of claim 5, wherein the distribution of color wavelengths comprises a first curve representing wavelengths detectable by S cone cells of the eye of the user, a second curve representing wavelengths detectable by M cone cells of the eye of the user, and a third curve representing wavelengths detectable by L cone cells of the eye of the user.

7. The non-transitory machine-readable storage medium of claim 1, wherein the instructions upon execution cause the controller of the wearable device to:
detect the color vision deficiency of the user based on identifying that a separation between peak portions of green and red spectral waveforms of the determined distribution of color wavelengths is less than a distance threshold.

8. The non-transitory machine-readable storage medium of claim 7, wherein the instructions upon execution cause the controller of the wearable device to:
identify a region of color confusion for the user, the region of color confusion comprising a range of wavelengths between the peak portions of the green and red spectral waveforms.

9. The non-transitory machine-readable storage medium of claim 8, wherein the instructions upon execution cause the controller of the wearable device to:
provide the control information by controlling a color filter of the display device based on the identified region of color confusion.

10. The non-transitory machine-readable storage medium of claim 9, wherein the controlling of the color filter comprises controlling a color filter comprising cholesterol liquid crystals.

11. The non-transitory machine-readable storage medium of claim 8, wherein the instructions upon execution cause the controller of the wearable device to:
adjust a hue of an image displayed by the display device based on the identified region of color confusion.

12. A wearable device comprising:
a display device; and
a controller to:
cause display of a test image in the display device;
adjust a hue of the displayed test image;
in response to an input provided by a user responsive to the displayed test image as the hue is adjusted, determine a distribution of color wavelengths for an eye of the user, the distribution of color wavelengths comprising a first spectral waveform for a green color detectable by the eye of the user as indicated by the input, and a second spectral waveform for a red color detectable by the eye of the user as indicated by the input;
detect a color vision deficiency of the user based on a distance between the first spectral waveform and the second spectral waveform; and
control the display device of the wearable device to compensate for the color vision deficiency.

13. The wearable device of claim 12, wherein the controller is to control the display device to compensate for the color vision deficiency by:
filtering out colors corresponding to a given range of wavelengths, or
adjusting a hue of an image displayed by the display device.

14. A method of a wearable device, comprising:
displaying a test image in a display device of the wearable device;
adjusting a hue of the displayed test image;
in response to an input provided by a user responsive to the displayed test image as the hue is adjusted, determining a distribution of color wavelengths for an eye of the user based on mapping information that maps hues to corresponding different distributions of color wavelengths;
detecting a color vision deficiency of the user based on the determined distribution of color wavelengths; and
controlling the display device of the wearable device to compensate for the color vision deficiency.

15. The method of claim 14, wherein the detecting of the color vision deficiency of the user is based on identifying that a separation between peak portions of green and red spectral waveforms of the determined distribution of color wavelengths is less than a threshold distance.
